(19) United States
(12) Patent Application Publication
(10) Pub. No.: US 2021/0315393 A1
(43) Pub. Date: Oct. 14, 2021

(54) Lateral Neck Support for Infants

(71) Applicant: Jami Meadows, New Braunfels, TX (US)

(72) Inventor: Jami Meadows, New Braunfels, TX (US)

(21) Appl. No.: 16/843,117

(22) Filed: Apr. 8, 2020

(51) Int. Cl. A47D 13/08; A41D 13/05

(52) U.S. Cl. CPC A47D 13/08; A41D 13/0512

(57) ABSTRACT

A lateral neck support meant to prevent an infant's head from tilting to one side in order to treat or prevent Torticollis and other abnormalities of the neck. The neck support is formed from soft pliable fabric including a somewhat cylindrical pillow-like support element in the center with a fabric strap extending from each end of the support element. The support element sits on top of the shoulder, beside the neck. The straps extend diagonally across the front and back of the torso and secure together under the opposite arm. The lateral neck support prevents undesirable head tilting to one side without being too restrictive for infant use.

### CROSS REFERENCE TO RELATED APPLICATION

This application claims the benefit of U.S. Provisional Application 62/815,388 entitled “LATERAL NECK SUPPORT”, filed Mar. 8, 2019 by the same inventor.

### FIELD OF INVENTION

The invention generally relates to neck supports. More specifically the invention relates to a lateral neck support adapted for infants, particularly to be used as an aide in treating and preventing Torticollis and other abnormalities of the neck.

### BACKGROUND

Torticollis is a common condition seen in babies. It can be present at birth (Congenital Muscular Torticollis CMT) due to in utero positioning or trauma caused to the neck during birth. It can also be acquired, due to postural or positional preferences or other medical conditions. Torticollis is a condition characterized by the tightening of the Sternocleidomastoid Muscle (SCM) on one side of the neck, causing the head to tilt to that side and limiting the range of motion in the neck. If the condition is left untreated it could cause developmental delays. It could also lead to the development of other conditions such as, but not limited to, Plagiocephaly. Plagiocephaly, or “Flat Head Syndrome” is characterized by a flattening of one area of the skull due to repeated pressure on one part of the head. The incidence of Torticollis and Plagiocephaly has increased since 1995 when the Academy of Pediatrics (AAP) started the “Back to Sleep Campaign”. In an effort to prevent Sudden Infant Death Syndrome (SIDS), it is now recommended that infants be put to sleep on their back instead of their stomach. This results in infants spending more time on their back, putting pressure on the back of the head and less time on their stomach, which is important for strengthening the developing neck muscles.

Typical treatment of Torticollis, as well as Plagiocephaly, begins with repositioning recommendations and physical therapy. It is recommended that parents limit the amount of time infants spend in carrier or container devices such as car seats, strollers, swings, bouncers and the like. The angle at which these carriers hold a baby can cause an infant with lack of head control to tilt and droop their head to one side. This contributes to the worsening of the Torticollis condition. Too much time spent in such carriers can impede recovery of Torticollis. Unfortunately, it is sometimes hard for parents to avoid use of these carriers as often as necessary. When infants with Torticollis are awake, the Stemocleidomastoid muscle (SCM) is actively tight. Usually, when the infant falls asleep the muscle will relax and allow the infant's head to move freely; however, it will most likely settle to the side that is tight. It would be desirable to have a lateral neck support that would prevent the infant's head from tilting to the side of the tight muscle and simultaneously encourage their head to tilt away from the support to allow for passive stretching of the tight muscle while the baby is asleep in the carrier. A variety of solutions have been proposed in prior art patent literature: U.S. Pat. No. 7,722,122 B2, issued May 25, 2010, U.S. Pat. No. 10,314,402 B2, issued Jun. 11, 2019, U.S. Pat. No. 9,968,198 B2, issued May 15, 2018, U.S. Pat. No. 9,408,471, issued Aug. 9, 2016 and U.S. Pat. No. 6,957,462 B1. The aforementioned prior art relates to head and neck supports meant for the comfort of an individual while trying to sleep in an upright position, primarily while traveling. Although the inventions of the aforementioned prior art would be suitable for the use of an adult or a child old enough to sit upright without help or supervision, they are not suitable for use by an infant that is not yet old enough to sit upright or without supervision. They would also be cumbersome for use in a car seat or similar type of carrier which holds the infant at an angle. The aforementioned inventions in the prior art would not be desirable or suitable for an infant with Torticollis because they are designed in such a way that the user finds it easy to lean or lay their head on the surface of the support structure. Therefore, the surface of the support structure would provide too adequate support for the infant's head and would not encourage the head to fall to the opposite side. Additionally, the prior inventions would interfere with an infant's ability to rotate their head in the direction of the support. For use by an infant with Torticollis, it would be better to have a support that only restricts movement in a way that prevents a certain undesirable position. Although the aforementioned inventions of the prior art include a strap type component that secures around the torso of the user which may allow it to be worn on the body, it would be better if the strap components were more infant friendly. Additionally, the aforementioned inventions of the prior art would not be suitable to be worn while doing any activity other than sitting and so would interfere with daily activities of the wearer.

It would be desirable to have a lateral neck support that did not restrict rotation of the head to either side but still prevented the head from tilting to the side of the tight muscle while simultaneously encouraging the head to tilt to the opposite side to allow for passive stretching of the tight muscle while the baby is asleep in the carrier. It would also be desirable to have lateral neck support designed to be worn on the body and constructed in such a way as to be worn for an extended period of time while the infant is awake, without interfering with the daily activities of the wearer.

Other prior art related to pillows: U.S. Pat. No. 8,813,283 B2, issued Aug. 26, 2014. This invention relates to a supplemental pillow that can be attached to a conventional pillow by means of a strap, meant to prevent the user's hair from making contact with the conventional pillow. U.S. Pat. No. 9,211,823 B2, issued Dec. 15, 2015, relates to a way to hold travel pillows. The two aforementioned inventions are not suitable for infant use and do not address the problems associated with Torticollis.

A variety of other solutions have been proposed in prior art patent literature: U.S. Pat. No. 7,566,313 B1, issued Jul. 28, 2009, U.S. Pat. No. 7,153,284 B2, issued Dec. 26, 2006, U.S. Pat. No. 8,096,304 B2, issued Jan. 17, 2012, and U.S. Patent Application Publication 2008/0184489. The aforementioned prior art is related to inventions that are to be worn on the head. The aforementioned prior art inventions treat Torticollis and Plagiocephaly together. They treat Torticollis by preventing the infant's head from settling in an undesirable position by preventing the infant from laying on certain spots of the head. While the aforementioned prior art inventions can be beneficial for treating Torticollis, they are not suitable for all patients. A device that addresses head shape is not necessary for an infant that has Torticollis but does not have Plagiocephaly. Additionally, the aforementioned inventions only address Torticollis if the infant has their head in contact with another surface. There remains a need for a lateral neck support that addresses undesirable head position without requiring the infant's head to be in contact with another surface.

A problem that caregivers face is maintaining the increased ranges of motion that are achieved after performing therapeutic stretches. It is a challenge to prevent an infant from returning to their preferred body position after a therapy session. It would be desirable to have a lateral neck support suitable for infants that would help prevent loss of progress between therapy sessions. Other solutions proposed in prior art: U.S. Pat. No. 2,801,630, issued Aug. 6, 1957, and U.S. Pat. No. 3,306,284, issued Feb. 28, 1967. The aforementioned prior art relates to cervical braces that encircle the neck completely and immobilize the neck to hold it in a desired position. While the aforementioned prior art inventions could help maintain progress made after performing therapeutic stretches, this type of brace is not suitable for infants as it is too restrictive. Limiting movement too much during infancy can negatively affect an infant's development. There remains a need for a lateral neck support suitable for infant use which helps maintain progress made from therapy that is minimally restrictive.

### SUMMARY OF THE INVENTION

The present invention is a lateral neck support suitable to be worn by an infant. It is a general object of the present invention to aide in the treatment or prevention of Torticollis and other abnormalities of the neck as well as Plagiocephaly which often coexists with Torticollis. The present invention is a simple device comprising an elevated center support roll or pillow, henceforth referred to as the support element, with a flat tab or strap extending off each end of the support element comprising of soft pliable fabric. The present invention is worn with the support element on top of the infant's shoulder beside the neck, on the side that is diagnosed with Torticollis (either left or right). The tabs that extend from each end of the support element and wrap diagonally around the front and back of the torso of the wearer and the ends of the tabs secure together under the arm opposite the support element. It is an object of the present invention to meet needs specific to an infant being treated for Torticollis. More specifically, it is an object of the present invention to prevent tilting of an infant's head without being overly restrictive. The support element should be large enough to hinder the infant from tilting the head toward the shoulder but should not block the infant's head from turning in the direction of the support element. The support element should not be large enough to push on the face and encourage the infant to look away from the pillow as this would be the directional position that Torticollis causes the infant to prefer. Encouraging the infant to turn the head in that direction would possibly increase the risk of the infant developing Plagiocephaly or contribute to worsening of said condition. The support element should be small enough for the infant to look over the support element by slightly lifting their chin when they look in the direction of the support element, if or when they are able. This action of lifting the chin when turning the head to look over the pillow causes the tight muscle to stretch and aides the therapeutic treatment. The support element should not be large enough or shaped in a way that would encourage the infant to rest or sleep with their head on the support element. This is why the design of current travel pillows are not suitable for use by an infant with Torticollis.

The support element should be sized in a way that it does not allow the infant to tilt their head to the side but also does not provide adequate support for the head if the infant falls asleep. Generally, when an infant with Torticollis falls asleep, the tight muscle in the neck will relax and allow the infant's head to move freely. If an infant falls asleep in their car seat, or a similar type of carrier while wearing the present invention, the support element should be sized so that it prevents the head from settling in a comfortable position on the side which it is being worn and so encourages the head to tilt to the opposite side for comfort. This allows the muscles in the infant's neck to stretch while the infant is sleeping. If not wearing the present invention, the tight neck muscle would pull the infants head further to the undesirable position. This position would likely cause the muscle to tighten again upon waking, and the infant could regress in treatment. When an infant is wearing the present invention, the caregiver will feel less guilty about the times their infant must be in a car seat or carrier.

It is an object of the present invention to aide in maintaining progress made during the treatment of Torticollis and prevent regression, and thus, reducing treatment time. Because the present invention is worn on the body with the support element against the neck, the infant does not have to be laying down or putting pressure on the head to correct the position of the head. Because the support element should not be cumbersome the present invention can be worn during all activities without interfering with daily life. Wearing the present invention for extended periods of time will prevent regression, thus improving recovery time. Improving recovery time will help prevent the infant from developing Plagiocephaly and other complications. It is another object of the present invention to be versatile in use. It may be beneficial for an infant to wear two of the present invention simultaneously. As a way to further promote correct body positioning when an infant is sitting in a carrier, an infant could wear one of the present invention with the support element on the shoulder as previously described, and also wear one of the present invention around the waist with the support element placed on the opposite side of the body above or on the hip with the tabs securing together on the opposite side of the body. An infant with a condition that causes weak muscles in the neck may benefit from wearing two of the present invention as a minimally restrictive way to support the head and neck in an upright position. This could be achieved by wearing one of present invention on each side of the neck. The following drawings will help with further understanding of the present invention.

### BRIEF DESCRIPTION OF THE DRAWINGS

FIG. 1 is a side view of the present invention

FIG. 2 is a view of the present invention with the side tabs attached

FIG. 3 is a view of the present invention being worn on the body.

FIG. 4 is a front view of the present invention

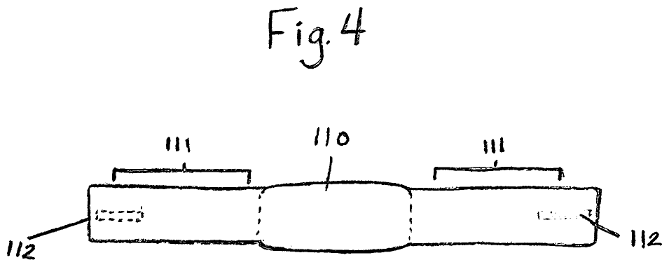

FIG. 5 shows an anticipated way to wear two of the present invention for body positioning purposes

FIG. 6 shows another anticipated way to wear two of the present invention

### DETAILED DESCRIPTION OF THE INVENTION AND DRAWINGS

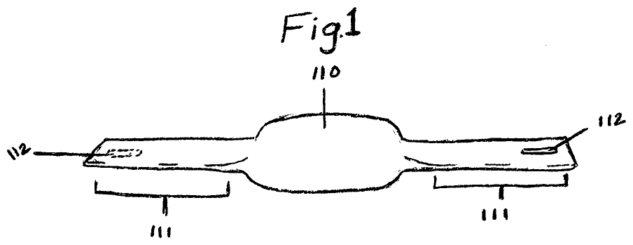

The present invention is a lateral neck support for infants. It is purposed to be worn by an infant to support one side of the neck. The present invention is designed to treat or prevent Torticollis and other abnormalities of the neck in infants. The present invention comprises of soft, pliable, possibly slightly stretchy decorative fabric, the long sides of the fabric are seamed together to form a tube-like structure. The center of the tube may be stuffed with natural filler or synthetic filler or rolled up batting or foam. The fabric is seamed on each end of the filler to hold the filler in place and form a somewhat cylindrical shaped support element 110. The size of the support element will vary according to the size requirement of the infant wearing it. The remaining fabric is sewn flat to form tabs or straps that extend from each end of the center support element 111. The length of the tabs will be determined by the size of the infant wearing it and should be approximately equal in length. The tabs should be long enough to fit snugly around the infant's torso. A fastening element of some kind, such as hook and loop, is attached to the distal end of each tab so the ends attach to each other 112. A side view of the present invention is shown in FIG. 1.

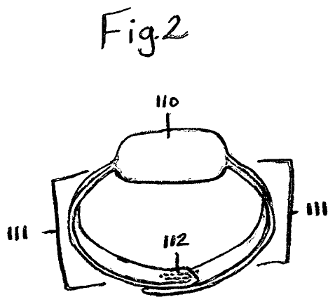

FIG. 2 shows a view of the present invention with the fabric tabs attached to each other 111. The side tabs should secure together in a way that allows them to overlap in an adjustable manner 112.

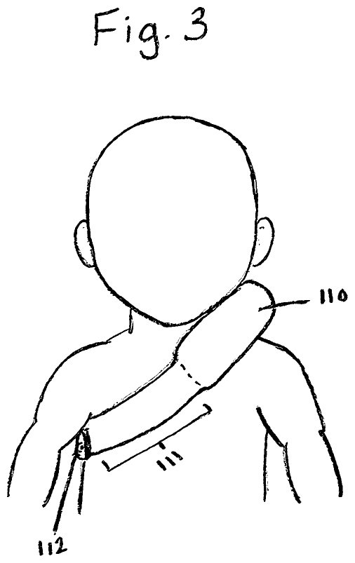

FIG. 3 shows a view of the present invention being worn by an individual with the support structure 110 on the shoulder beside the neck and the fabric tabs 111 diagonally crossing the body and secured 112 together under the arm. The present invention could also be worn on the other side of the body.

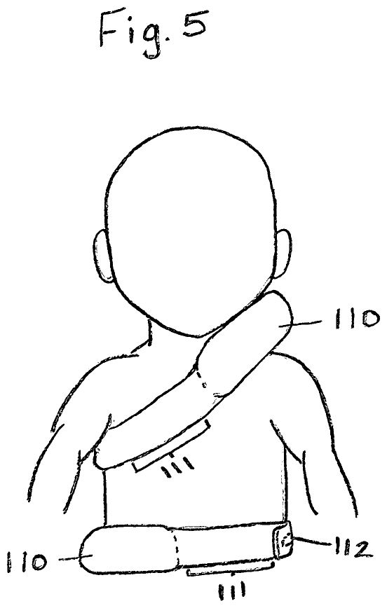

FIG. 5 shows an anticipated way to wear two of the present invention as to further reposition the body of the wearer if further repositioning is necessary. This is to demonstrate versatility of the present invention. Physical therapy treatment for Torticollis includes positioning recommendations. In an infant who has not yet developed adequate muscle strength, tilting the head to the side can cause the entire spine to curve causing asymmetry of the body. There may be times that it is beneficial to wear two of the present invention at the same time with one around the waist and as well one on the shoulder. Wearing two of the present invention as shown in FIG. 5 can promote proper body posture of an infant.

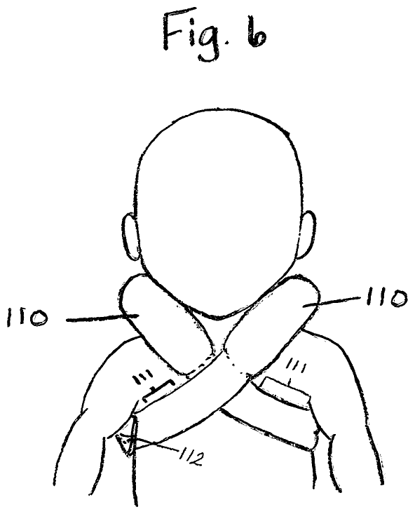

FIG. 6 shows another anticipated way to wear two of the present invention. Torticollis is not the only condition in which an infant suffers from abnormalities of the neck. Infants with other conditions may benefit from wearing two of the present invention together by placing one support element 110 on each side of the neck and overlapping the fabric tabs 111 in an “X” pattern around the torso. Wearing two of the present invention as shown in FIG. 6 is a minimally restrictive, gentle way to support the head of an infant in an upright position. Wearing two of the present invention in this way could be beneficial for an infant who suffers from low muscle tone.

What is claimed:

1. A lateral neck support meant to prevent an infant's head from tilting to one side in order to treat or prevent Torticollis and other abnormalities of the neck, comprising: soft pliable fabric sleeve including a somewhat cylindrical pillow-like support element in the center with the remainder of said fabric extending from each end forming elongated tabs that secure together by means of a fastening element by overlapping the ends of said tabs.

2. The lateral neck support of claim 1 wherein: said support element comprises a pillow body comprising natural filler, foam, or synthetic filler, having a seam to the right and left of the filler to hold the filler in place therefore forming the pillow-like support element.

3. The lateral neck support of claim 1 wherein: said lateral neck support comprise of two elongated flat tabs formed from said fabric that extend, one from each end of said support element, long enough to encircle an infant's torso.

4. The tabs of claim 3 wherein: said tabs comprise a hook and loop fastener element near the distal end of each of said tabs that allows for adjustment when said tabs are attached.

5. A method of treating and preventing Torticollis and other abnormalities of the neck comprising:
A lateral neck support suitable for use by and infant that can be worn on the body for extended periods of time, comprising of soft pliable fabric comprising a center support element that is worn on an infant's shoulder, next to the neck and held in place by tabs which extend from each end of the support element and attach together under the infant's opposite arm by an adjustability enabling fastening element, wherein the support element is suitable in size to prevent an infant from tilting their head without restricting movement of the head in an undesirable way and has the ability to be used in pairs to promote correct body positioning.
